(19) United States
(12) Patent Application Publication
(10) Pub. No.: US 2025/0082465 A1
(43) Pub. Date: Mar. 13, 2025

(54) TAVR Commissure Design

(71) Applicant: St. Jude Medical, Cardiology Division, Inc., St. Paul, MN (US)

(72) Inventors: Zachary R. Vidlund, Robbinsdale, MN (US); Prem Midha, St. Paul, MN (US); Brandon Moore, St. Louis Park, MN (US); Jay Reimer, Shoreview, MN (US); Mai Moua, Circle Pines, MN (US); Somnhot Thamlansy, New Hope, MN (US); Victoria Schuman, Minneapolis, MN (US); Kent J. Smith, Shoreview, MN (US); Janet Glenn, Forest Lake, MN (US); Sarah Tanck, Green Bay, WI (US)

(73) Assignee: St. Jude Medical, Cardiology Division, Inc., St. Paul, MN (US)

(21) Appl. No.: 18/804,723

(22) Filed: Aug. 14, 2024

Related U.S. Application Data
(60) Provisional application No. 63/582,061, filed on Sep. 12, 2023.

(51) Int. Cl. A61F 2/24; A61F 2/00

(52) U.S. Cl. CPC A61F 2/2409; A61F 2002/0081; A61F 2220/0075; A61F 2230/0019

(57) ABSTRACT

In some embodiments, a prosthetic heart valve system includes a stent having a plurality of commissure attachment features, a cuff coupled to the stent, and a plurality of leaflets, the cuff and the plurality of leaflets forming a valve assembly, each of the plurality leaflets having two leaflet attachment regions, the leaflet attachment regions being folded adjacent the plurality of commissure attachment features.

### CROSS-REFERENCE TO RELATED APPLICATIONS

The present application claims priority to U.S. Provisional Ser. No. 63/582,061, filed Sep. 12, 2023, the disclosure of which is hereby incorporated by reference in its entirety as if fully set forth herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”'s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets, and may also include a cuff or skirt. The cuff may be disposed on the stent's interior or luminal surface, its exterior or abluminal surface, and/or on both surfaces. A cuff helps to ensure that blood does not flow around the valve leaflets if the valve or valve assembly is not optimally seated in a valve annulus. A cuff, or a portion of a cuff, disposed on the exterior of the stent can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When self-expandable prosthetic heart valves are delivered into a patient to replace a malfunctioning native heart valve, the self-expandable prosthetic heart valve is almost always maintained in the collapsed condition within a capsule of the delivery device. While the capsule may ensure that the prosthetic heart valve does not self-expand prematurely, the overlying capsule (with or without the help of additional internal retaining features) helps ensure that the prosthetic heart valve does not come into contact with any tissue prematurely, as well as helping to make sure that the prosthetic heart valve stays in the desired position and orientation relative to the delivery device during delivery. However, balloon expandable prosthetic heart valves are typically crimped onto the balloon of a delivery device without a separate capsule that overlies and/or protects the prosthetic heart valve. One reason for this is that space is always at a premium in transcatheter prosthetic heart valve delivery devices and systems, and adding a capsule in addition to the prosthetic valve and the underlying balloon may not be feasible given the size profile requirements of these procedures.

The continuous opening and closing of a prosthetic heart valve may result in mechanical stresses on the leaflet tissue (e.g., the leaflet attachment regions), which can affect performance and longevity of the valve. Specifically, the leaflet attachment region, which is coupled to the commissure attachment features may experience high stress during the repeated movement of the leaflets. Among other advantages, it would be beneficial to provide new prosthetic heart valve configurations with features for managing the high stresses in this region.

### BRIEF SUMMARY OF THE DISCLOSURE

In some embodiments, a prosthetic heart valve system includes a stent having a plurality of commissure attachment features, a cuff coupled to the stent, and a plurality of leaflets, the cuff and the plurality of leaflets forming a valve assembly, each of the plurality leaflets having two leaflet attachment regions, the leaflet attachment regions being folded adjacent the plurality of commissure attachment features.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIGS. 2A-B illustrates a prosthetic heart valve PHV, crimped over a balloon in the deflated and inflated conditions.

FIGS. 3A-C illustrate a first embodiment showing the attachment of a leaflet to an elongated commissure attachment feature.

FIGS. 4A-C illustrate a second embodiment showing the attachment of a leaflet to a rectangular commissure attachment feature having slots.

FIGS. 5A-B illustrate a third embodiment showing the attachment of a leaflet directly to stent nodes.

FIGS. 6A-D illustrate a fourth embodiment showing the attachment of a leaflet to a rectangular commissure attachment feature having two slots.

FIGS. 7A-B illustrate a fifth embodiment showing the attachment of a leaflet to a commissure attachment feature having three slots.

FIGS. 8A-E illustrate a sixth embodiment showing the attachment of a leaflet to a commissure attachment feature with variable takeoff angles.

FIGS. 9A-B illustrate a seventh embodiment showing the attachment of a leaflet to a commissure attachment feature with fabric buffer.

FIGS. 10A-B illustrate an eighth embodiment showing the attachment of a leaflet to a fabric cuff having a window.

FIGS. 11A-B illustrate a ninth embodiment showing the attachment of a leaflet to a rectangular commissure attachment feature.

FIGS. 12A-B illustrate a tenth embodiment showing the attachment of a leaflet to a single strut.

FIGS. 13A-B illustrate an eleventh embodiment showing the attachment of a leaflet to a Π-shaped commissure attachment feature.

FIGS. 14A-B illustrate a twelfth embodiment showing the attachment of a leaflet to a X-shaped commissure attachment feature.

FIGS. 15A-D illustrate a thirteenth embodiment showing the attachment of a leaflet via scrolling of the leaflet attachment regions.

FIG. 16 illustrates a fourteenth embodiment showing a commissure attachment feature having two parallel struts unattached at the top.

FIGS. 17A-B illustrate a fifteenth embodiment showing the folding of a leaflet attachment region to increase compliance.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation, but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to a trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

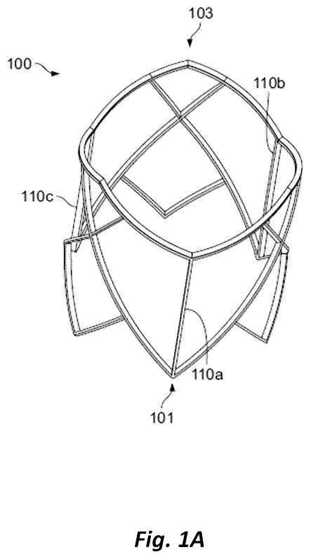

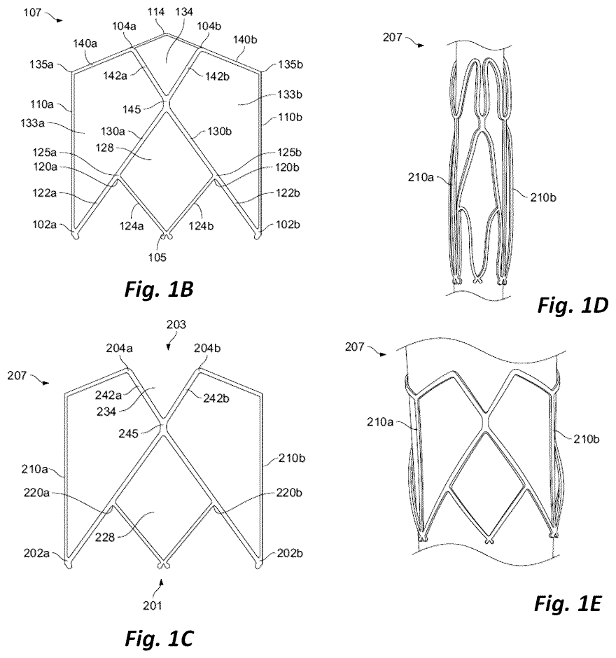

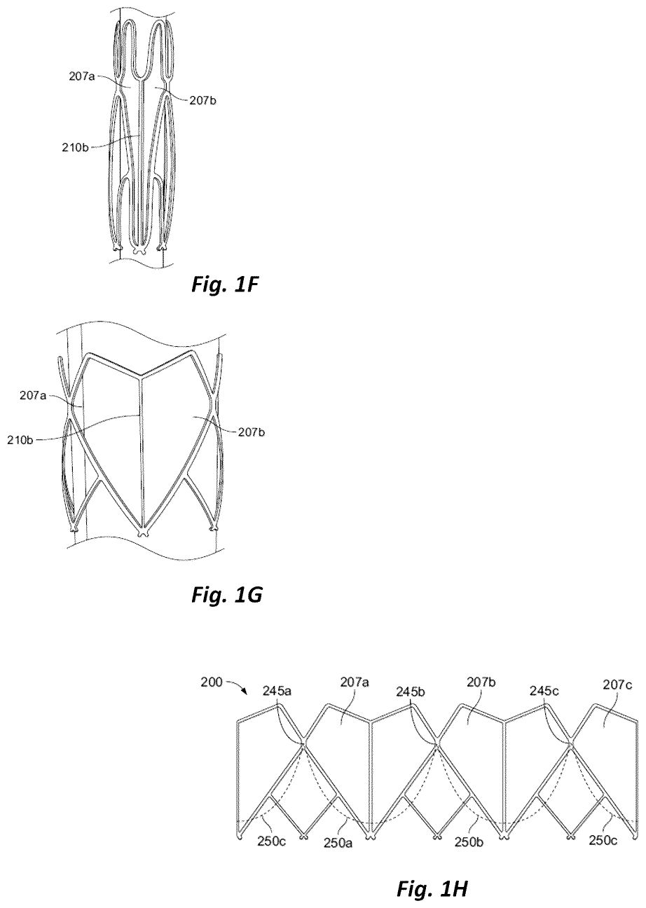

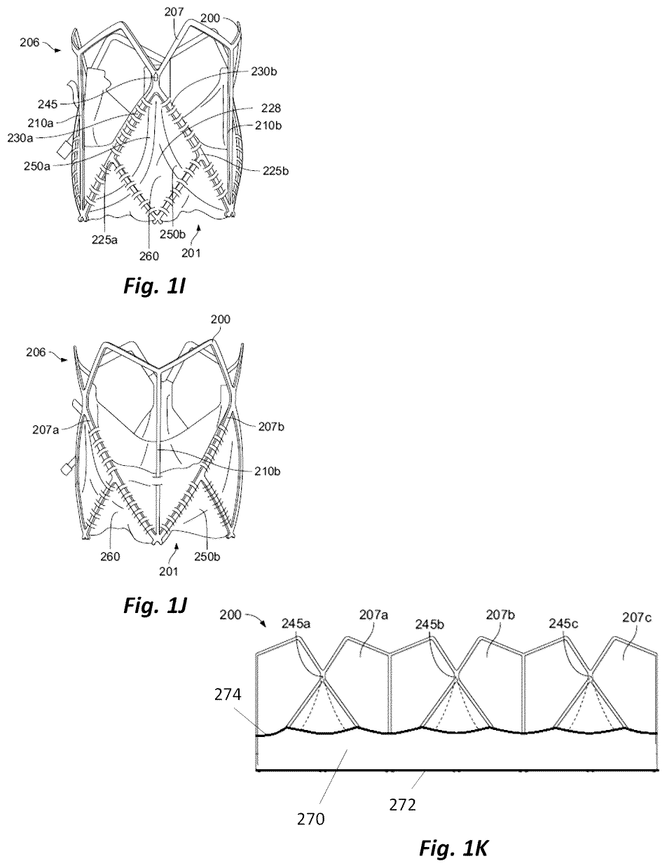

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100, and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135a and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g., wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

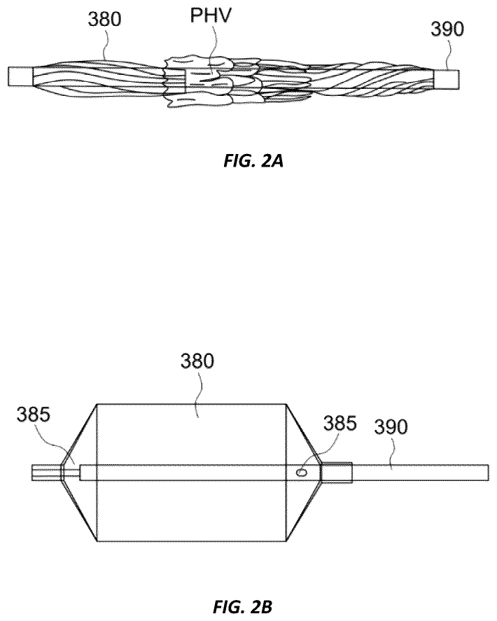

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

The present disclosure provides several techniques, devices and methods to reduce stresses in a leaflet material by allowing reduction, redistribution and/or dampening of stresses during valve operation. The devices, methods and techniques provided herein relate to unique attachment methods between the leaflets, cuffs, stents, buffers, and/or commissure attachment features adjacent high stress regions of the valve. While several embodiments are shown, it will be appreciated that certain features may be combined with one another.

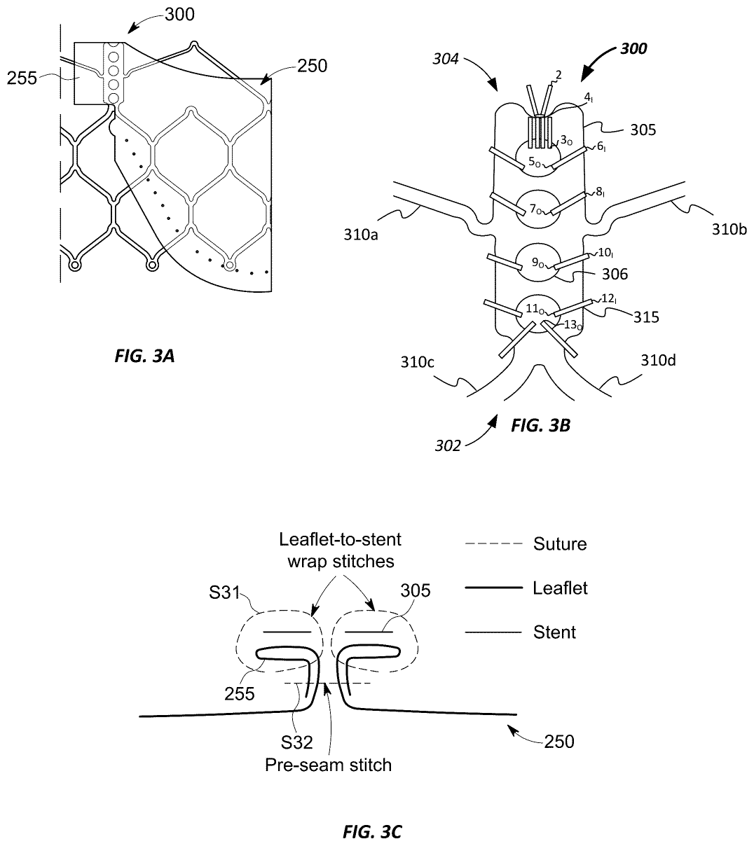

FIGS. 3A-C show a first commissure attachment feature 300 being attached to a leaflet attachment region 255 of a leaflet 250. As shown in greater detail in FIG. 3B, commissure attachment feature 300 extends between a proximal end 302 and a distal end 304. In this embodiment, commissure attachment feature 300 includes a generally elongated body 305 defining a number of eyelets 306 (e.g., two, three, four or more eyelets). Commissure attachment feature 300 may be integrally formed with, or coupled to, a stent via a number of struts. As shown, commissure attachment feature 300 is coupled to two lateral struts 310a-310b, and two lower struts 310c-310d disposed adjacent proximal end 302. The distal end of commissure feature 300 is free or unattached to other stent components. Commissure attachment feature 300 may be coupled to one or more leaflets via sutures 315. FIG. 3B is an outer diameter view, and certain annotations are made to show the order in which the suture is passed in and out of the leaflet. For example, notations 41, 61, 81, 101 and 121 indicate a suture being passed in toward the inner diameter and through the leaflet, while notations 30, 50, 70, 90, 110 and 130 indicate a suture being passed out of the leaflet toward the outer diameter. The numbering in the notations generally shows the order in which the suture is passed at certain locations. Approximately only half of the suture positions are annotated, and it will be understood that the pattern is generally symmetric about the longitudinal axis. In some examples, leaflet attachment region may be folded on the inner diameter of the stent, as shown in the top-down cross-sectional view of FIG. 3C, for added structural integrity and sutures S31 may be looped around the folded edges of the tissue and through the holes in commissure attachment feature 300 (shown as the gap between black portions of the leaflet). An additional suture S32 may couple the two leaflets together. It will be understood that sutures S31 and S32 may be formed as a continuous suture, or as discrete portions.

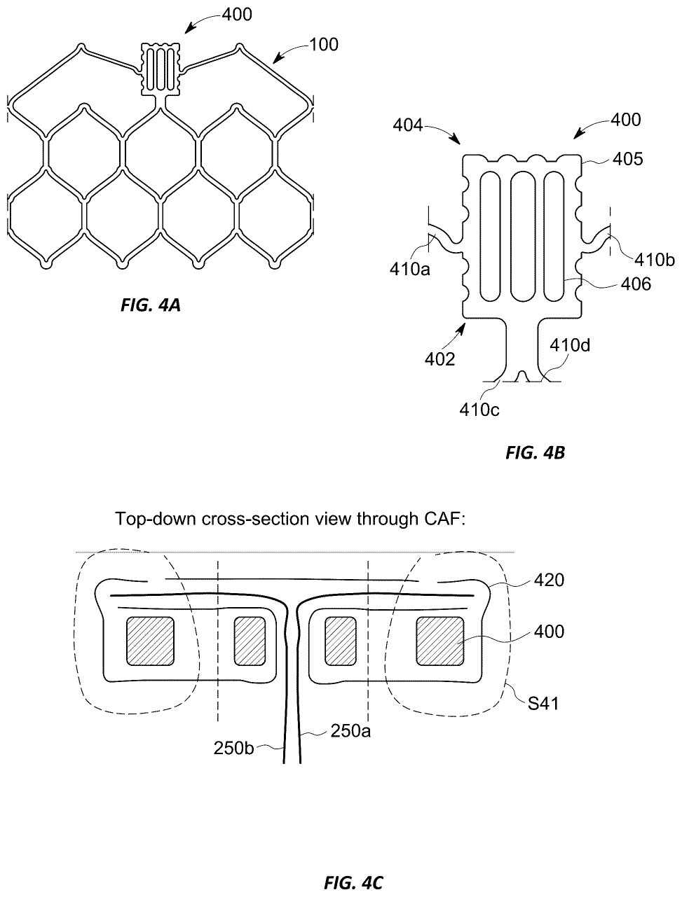

FIGS. 4A-C show another commissure attachment feature 400 being integrally formed with, or attached to, a stent 100. Commissure attachment feature 400 may be attached to a leaflet attachment region of a leaflet. As shown in greater detail in FIG. 4B, commissure attachment feature 400 extends between a proximal end 402 and a distal end 404. In this embodiment, commissure attachment feature 400 includes a generally elongated body 405 defining a number of longitudinally-extending slots 406. In this example, three slots are defined, the three slots being of generally the same shape and size. It will be understood that any number of slots may be formed, and that the slots may be of different shapes and/or sizes. Commissure attachment feature 400 may be integrally formed with, or coupled to, a stent via a number of struts. As shown, commissure attachment feature 400 is coupled to two lateral struts 410a-410b, and two lower struts 410c-410d disposed adjacent proximal end 402. The distal end of commissure feature 400 is free or unattached to other stent components.

As best shown in the top-down cross-sectional view of FIG. 4C, commissure attachment feature 400 may be coupled to one or more leaflets 250a,250b via sutures S41. Specifically, leaflets 250a,250b are passed through the central slot of the commissure and lay over the outer diameter of commissure attachment feature 400 to distribute stress over a surface area, rather than point-loading the stress at suture attachments. In this example, one or more reinforcing buffer strip(s) 420 may be wrapped around portions of the commissure attachment feature 400 to help sandwich the leaflets(s) and protect them from abrasion from pressing against stent. In the example shown, three discrete buffer strips 420 are used, although it will be understood that a single continuous buffer strip, or two or more buffer strips are possible. In some examples, buffer strips 420 include a fabric, or a biological material.

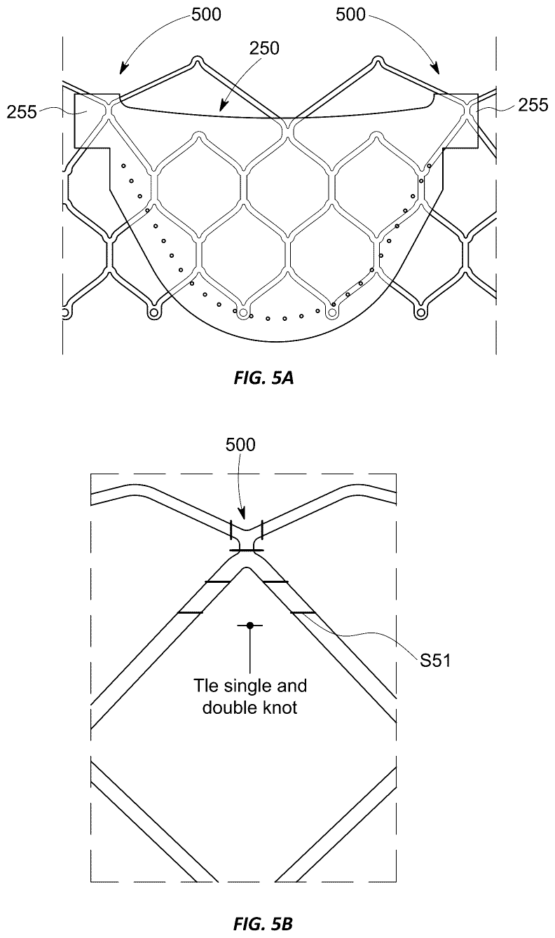

Though the previous embodiments have shown stents with clearly defined commissure attachment features, attachment of leaflets may be possible without such features. For example, in FIGS. 5A-B another embodiment is shown where no separate commissure attachment features are formed. Rather, the embodiment shows a simplified stent design that maintains structural integrity of the leaflets and their attachment. As shown, leaflet attachment regions 255 of the leaflets 250 are stitched directly to stent struts and stent nodes 500 where certain struts intersect. As shown in the detailed view of FIG. 5B, a symmetric stitching pattern is used to couple the leaflet attachment regions 255 to stent nodes 500 via suture pattern S51. The overall bulk of the prosthetic heart valve may be reduced if secure attachment of the leaflets can be made to the nodes without separate commissure attachment features.

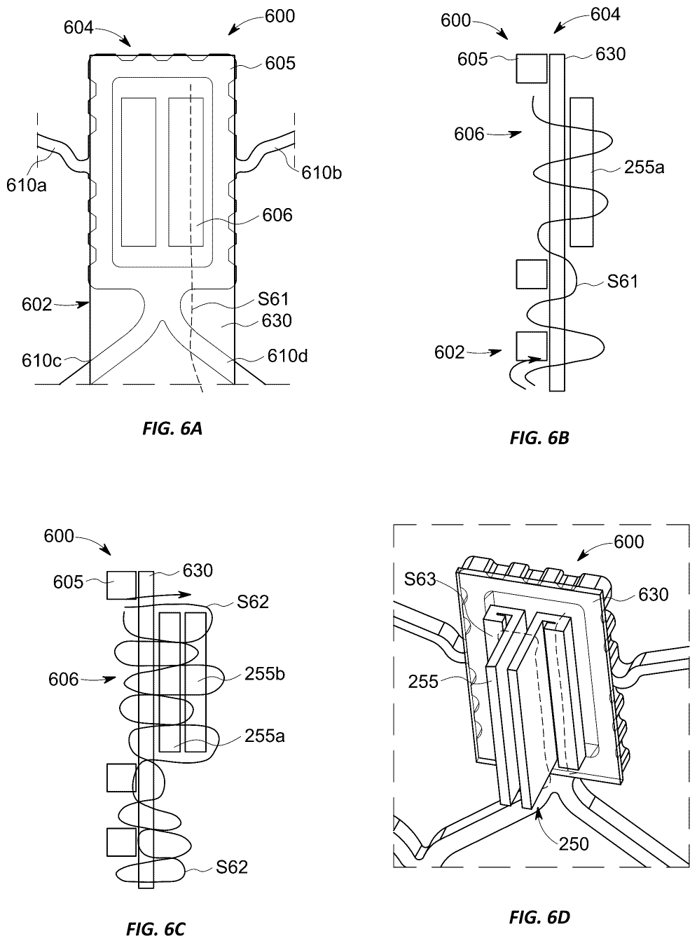

FIGS. 6A-D show another commissure attachment feature 600 being integrally formed with, or attached to, a stent. Commissure attachment feature 600 may be attached to a leaflet attachment region of a leaflet. As shown in FIG. 6A, commissure attachment feature 600 extends between a proximal end 602 and a distal end 604. In this embodiment, commissure attachment feature 600 includes a generally elongated body 605 defining a pair of longitudinally-extending slots 606. In this example, two rectangular slots are defined of generally the same size. Commissure attachment feature 600 may be integrally formed with, or coupled to, a stent via a number of struts. As shown, commissure attachment feature 600 is coupled to two lateral struts 610a-610b, and two lower struts 610c-610d disposed adjacent proximal end 602. The distal end of commissure feature 600 is free or unattached to other stent components.

Two leaflet attachment regions 255 belonging to two distinct leaflets 250 may be coupled to each commissure attachment feature 600. FIG. 6B shows the coupling of one leaflet attachment region 255a to a commissure attachment feature 600. As shown, a rectangular reinforcing swatch 630 may be placed on the luminal surface of the commissure attachment feature 300 and attached thereto with sutures or other appropriate coupling means. Reinforcing swatch 630 be formed of fabric, or any of the materials of the leaflets or cuff (e.g., a biological material, such as animal pericardium, or synthetic materials, such as ultra-high molecular weight polyethylene).

The leaflet attachment region 255 may approach the commissure attachment feature at a 90-degree angle and bend so that a first portion 255a of the leaflet attachment feature is parallel with reinforcing swatch 630 and commissure attachment feature 600 (See, FIG. 6D). Sutures may attach leaflet attachment regions to the reinforcing swatch 630. Specifically, in some embodiments, the same suture that is used to attach the leaflet belly is also used to attach the leaflet attachment regions at the commissure attachment features 300. Turning back to FIG. 6B, beginning adjacent proximal end 602 and extending toward distal end 604, a first suture pattern S61 (which may be the same running stich from the belly attachment) may travel in and out of reinforcing swatch 630 and the leaflet attachment region 255a as shown. Notably, leaflet attachment region 255a is only shown being directly coupled to reinforcing swatch 630, and not directly to the commissure attachment feature 600 or any other stent element to add compliance at the commissure and produce a “drum effect.” As shown in FIG. 6C, when first suture patterns S61 arrives at the top of the commissure attachment feature 600, leaflet attachment region 255 may be folded over itself to form a second portion 255b, and a second suture pattern S62 may create a reverse running stitch that extends from the distal end 604 to the proximal end 602. It will be understood that in some embodiments, first sutures pattern S61 and second suture pattern S62 are formed of a single continuous suture, though other variations are possible. Upon completion of the suture patterns, a leaflet attachment region 255 of a first leaflet will be coupled to reinforcing swatch 630 at one of the slots 606 of the commissure attachment feature 600. A second leaflet may be disposed and coupled as a mirror-image of the first leaflet, and the two leaflets may be attached to one another with a third cinching suture S63.

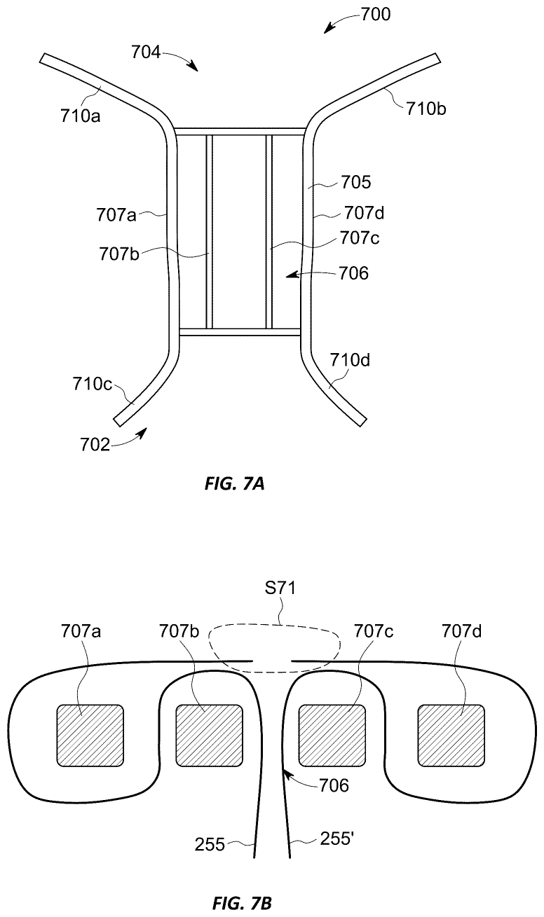

FIGS. 7A-B show another commissure attachment feature 700 for securing a leaflet attachment region of a leaflet. As shown in FIG. 7A, commissure attachment feature 700 extends between a proximal end 702 and a distal end 704. In this embodiment, commissure attachment feature 700 includes a generally rectangular body 705 defining three longitudinally-extending slots 706 defined by four parallel beams 707a-707d. Commissure attachment feature 700 may be integrally formed with, or coupled to, a stent via a number of struts. As shown, commissure attachment feature 700 is coupled to two upper struts 710a-710b at distal end 704, and two lower struts 710c-710d adjacent proximal end 702.

Turning to FIG. 7B, two leaflet attachment regions 255,255′ belonging to two distinct leaflets 250 may be coupled to each commissure attachment feature 700. To distribute stress over a surface area (rather than point-loading at suture attachments), portions of a leaflet may be passed through the elongated slots 706 between beams 707a-707d and wrapped on the outer diameter of commissure attachment feature 700. Specifically, a first attachment regions 255 may pass through the central slot 706 between beams 707b and 707c, then bend and pass through a peripheral slot between beams 707a and 707b, then wrap around beam 707a to end on the abluminal surface of commissure attachment feature 700. Similarly, an opposing attachment region 255′ may pass through the central slot 706 between beams 707b and 707c, then bend and pass through a peripheral slot between beams 707c and 707d, then wrap around beam 707d to end on the abluminal surface of commissure attachment feature 700. The two free tails of attachment regions 255,255′ may be coupled together with a suture S71. Using these techniques, it is believed that multiple wraps around the commissure attachment feature 700 will result in friction holding the attachment regions in place so that minimal stitching is needed.

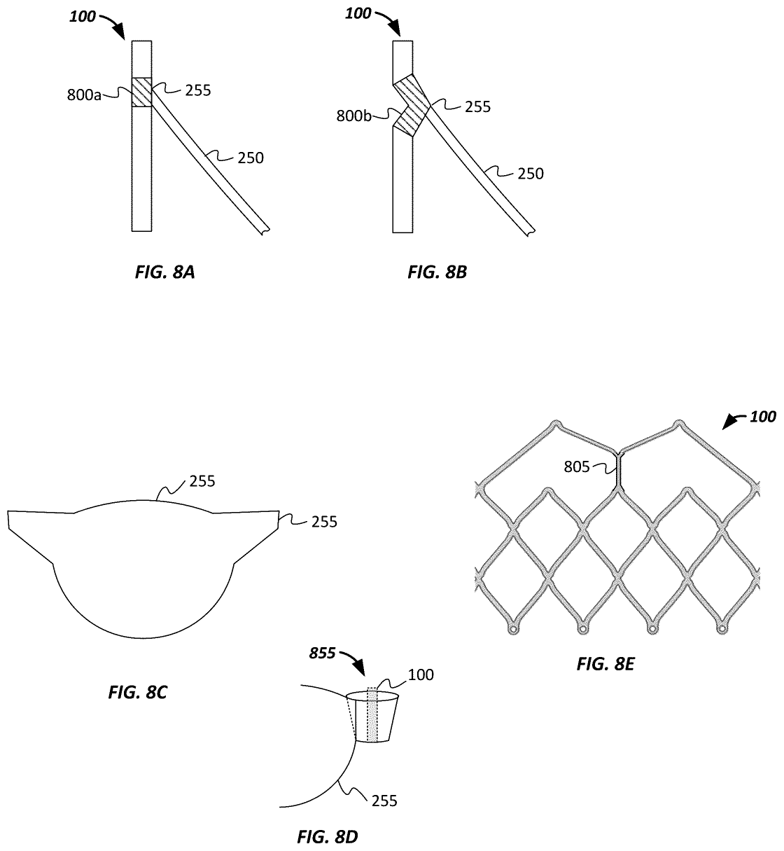

FIGS. 8A-B illustrate another technique to reduce the stress on the leaflet, and specifically on the leaflet attachment region. As shown in FIG. 8A, conventional stents 100 may include an in-line commissure attachment feature 800a that is coupled to leaflet attachment region 255. Conversely, as shown in FIG. 8B, a stent 100 may be formed with a mis-aligned commissure attachment feature 800b. The mis-alignment of commissure attachment feature 800b may include inwardly tilting or bending a portion of the stent so that commissure attachment feature 800b defines a smaller diameter than other portions of the stent. Modifying commissure attachment feature 800b in this manner may reduce the takeoff angle of the leaflet and reduce stresses on the leaflet attachment region. This may also better align the direction of the free edge of the leaflet, in the closed configuration, with the direction of the force vector from blood pressure loading. In another example, shown in FIGS. 8C-E, a leaflet 255 may be manufactured to include a tabbed leaflet attachment region 255 configured to provide more give at the top and to be slightly tighter at the bottom. The leaflets 255 may be attached to a stent (FIG. 8E) so that the tabbed leaflet attachment region 255 can be bent, wrapped and/or rolled into a cone shape 855 around a portion of a stent (e.g., a strut, node, commissure attachment feature, etc.) to attach the leaflets (FIG. 8D). As shown, the tabbed leaflet attachment region 255 will be wrapped around a single strut 805 shown in FIG. 8E.

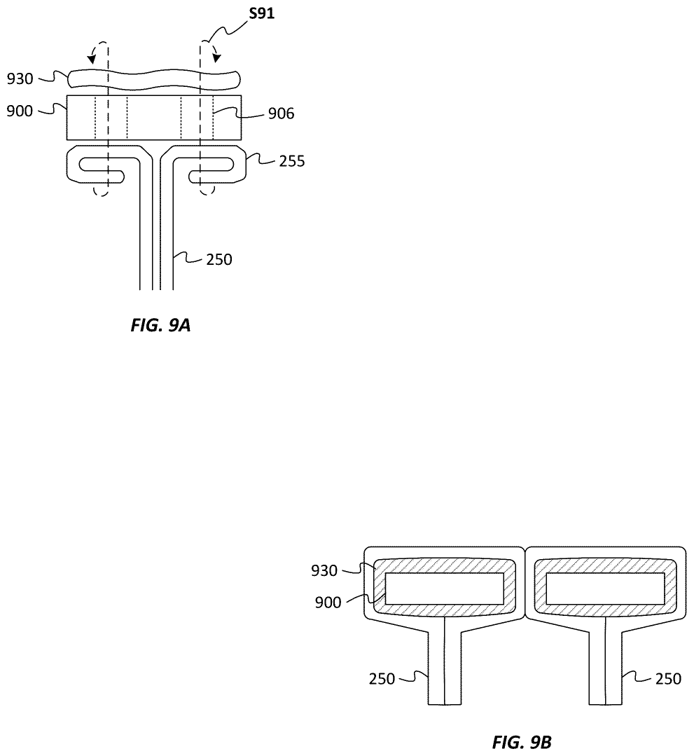

FIG. 9A illustrates an embodiment in which compliance is increased by introducing a relatively-thin compressible material 930 to the abluminal (or luminal) surface of a commissure attachment feature 900 having three slots 906 similar to the commissure attachment feature described above with reference to FIG. 7A. Specifically, compressible material 930 may include a rubbery, squishy or compliant material to reduce the stresses in these areas. Compressible material 930 may be formed of a fabric, a rubber, or any of the materials of the leaflets or cuff (e.g., a biological material, such as animal pericardium, or synthetic materials, such as ultra-high molecular weight polyethylene). As shown, a pair of leaflets 250, each with a leaflet attachment region 255, is disposed opposite compressible material 930. Leaflet attachment regions 255 are folded similar to the embodiment described with reference to FIGS. 6A-D. A suture pattern S91 couples each of the leaflet attachment regions 255 through slots of the commissure attachment features 900 to the compressible material 930. Suture pattern S91 may extend through the commissure attachment features 900 but not directly couple thereto, so that the commissure attachment feature is not directly coupled to the leaflets. Alternatively, the suture pattern S91 may be modified so that the suture wraps around one or more of the bars of the commissure attachment feature 900.

In another example, instead of being disposed only on the abluminal surface of commissure attachment feature 900, a compliant material 930 may be wrapped around a bar of a commissure attachment feature 900 (or strut of the stent) as shown in FIG. 9B, and the leaflet attachment region 255 may be wrapped around the compliant material 930 so that the compliant material provides a buffering effect. Compliant material 930 may include a viscoelastic material, a memory foam, or a fluffy cuff material.

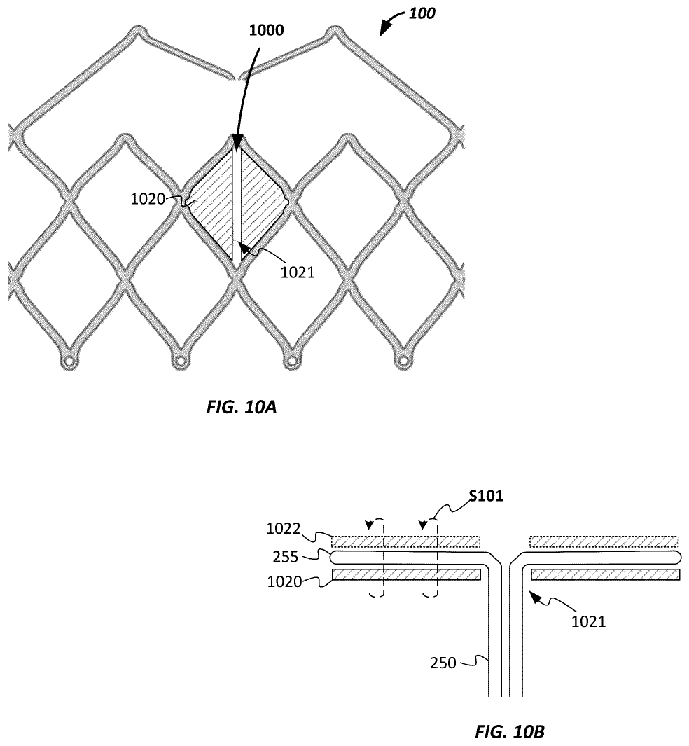

FIGS. 10A-B illustrate an embodiment in which stress is distributed over a surface by passing a leaflet through a fabric and wrapping it around the outer diameter of the fabric. As shown, a prosthetic heart valve 100 may include a cuff 1020 (e.g., an inner fabric cuff) covering certain cells. Cuff 1020 may be disposed over only a selected cell, or may be disposed over rows or cells on the luminal or abluminal surface of the stent. Cuff 1020 may include a slit 1021 configured and arranged to receive a leaflet attachment region 255 of a leaflet 250. As shown in FIG. 10B, portions of two leaflets 250 may pass through each slit 1021 of the cuff, and wrap around the outer diameter of the cuff 1020 before being coupled to the cuff 1020 via one or more sutures S91. Optionally, a second outer layer of fabric 1022 may be used to create a fabric sandwich around the leaflet attachment region 255 of each leaflet.

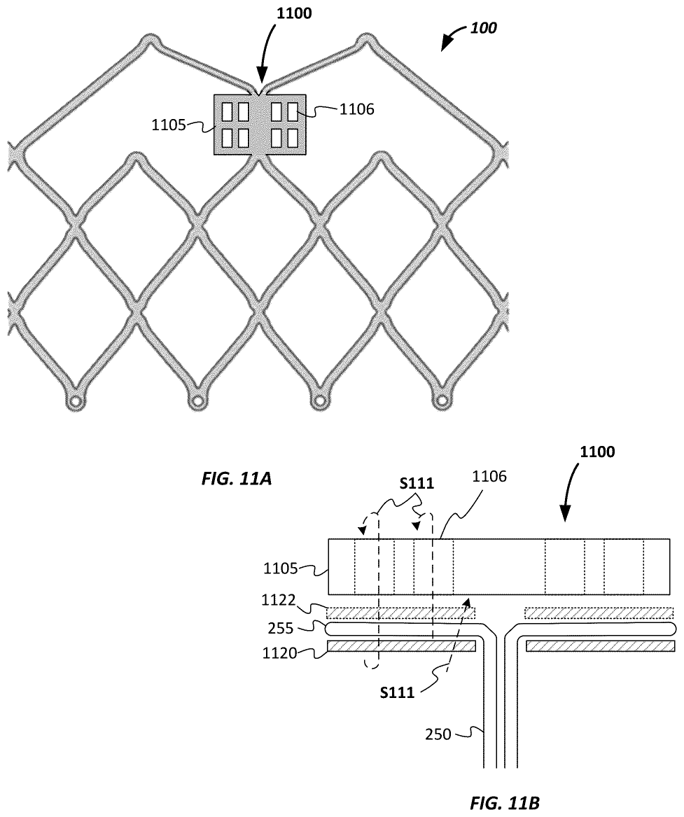

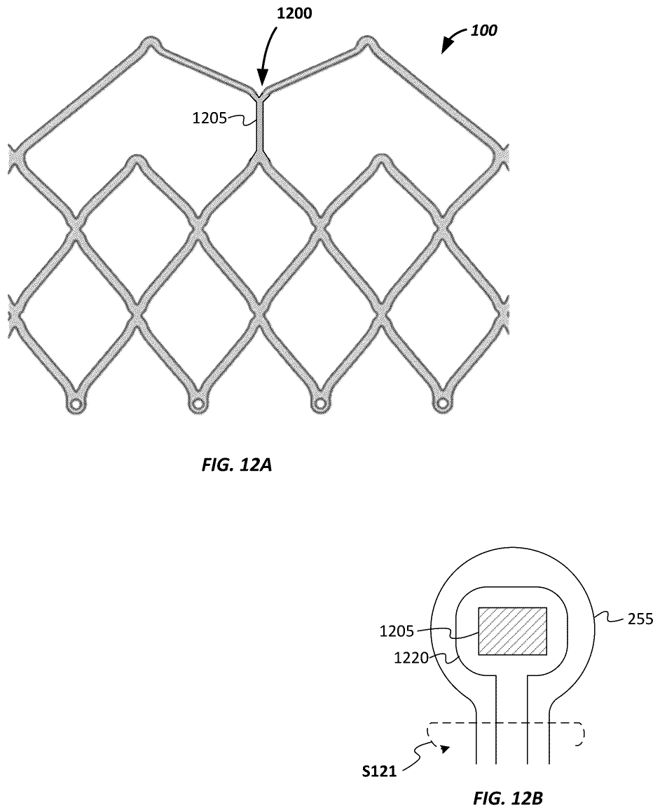

FIGS. 11A-B illustrate an embodiment in which stress is distributed over a surface by sandwiching a leaflet via fabrics 1120,1122. As shown, a prosthetic heart valve 100 includes a rectangular commissure attachment feature 1100 having a body 1105 defining eyelets 1106. In this example, eight rectangular eyelets 1106 are arranged to accept sutures to couple leaflets to commissure attachment feature 1100. As shown in FIG. 11B, in addition to being coupled to commissure attachment feature 1100 via sutures S111, the leaflets 250, and specifically the leaflet attachment region 255 may be placed adjacent, and coupled to, one or more fabrics 1120,1122. It will be understood that either fabric may be used alone, or that both fabrics may be used. Examples are also possible where no fabric at all is used. The fabrics may be placed on the luminal surface, the abluminal surface or a combination. In this example, certain sutures S111 couple the leaflet attachment regions 255, the fabrics 1120,1122 and the commissure attachment features 1100. Other sutures S112 may couple the commissure attachment features 1100, the fabric 1122 and the leaflet attachment region 255. Other sutures S113 may couple the fabrics 1122, 1120 and the leaflet attachment region 255 only. Variations and combinations of these suture attachments are possible.

FIGS. 12A-B show an embodiment where a stent 100 includes a commissure attachment features 1200 in the form of a single strut 1205. In this example, a fabric 1220 may be wrapped around the single strut 1205 to provide a buffer, and the leaflet attachment region 255 may be wrapped around the fabric 1220. A suture S121 may secure leaflet attachment region 255 and the fabric 1220 to the single strut 1205.

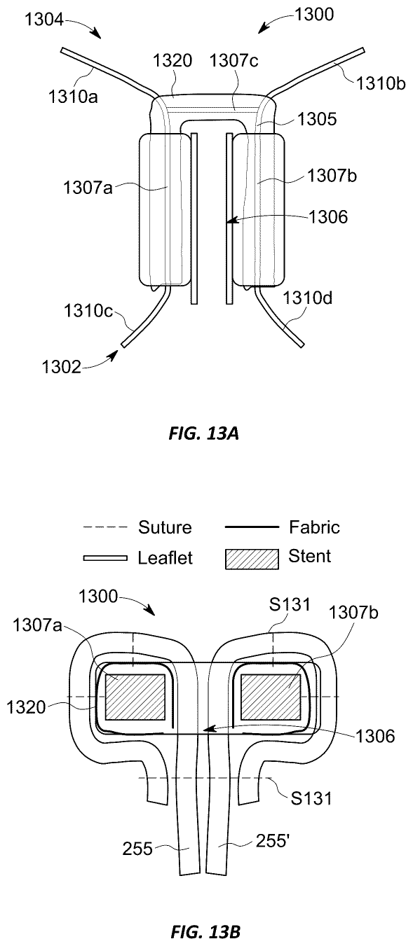

FIGS. 13A-B show another commissure attachment feature 1300 for securing a leaflet attachment region of a leaflet. As shown in FIG. 13A, commissure attachment feature 1300 extends between a proximal end 1302 and a distal end 1304. In this embodiment, commissure attachment feature 1300 includes a generally Π-shaped body 1305 with a slot 1306 defined by two parallel beams 1307a, 1307b and a top beam 1307c. Commissure attachment feature 1300 may be integrally formed with, or coupled to, a stent via a number of struts. As shown, commissure attachment feature 1300 is coupled to two upper struts 1310a-1310b at distal end 1304, and contiguously forms two lower struts 1310c-1310d adjacent proximal end 1302. A horseshoe-shaped scroll of fabric 1320 may cover the side beams 1307a, 1307b and top beam 1307c.

Turning to FIG. 13B, two leaflet attachment regions 255,255′ belonging to two distinct leaflets may be coupled to each commissure attachment feature 1300. To distribute stress over a surface area (rather than point-loading at suture attachments), portions of a leaflet may be passed through the elongated slots 1306 between beams 1307a, 1307b and wrapped around the corresponding struts 1307a, 1307b to form a complete revolution. Optionally, each of beams 1307a, 1307b may be wrapped with a fabric 1320 so that the rigid stent structures do not directly touch the leaflet attachment regions 255. Sutures S131 may secure leaflet attachment regions 255,255′ together as shown.

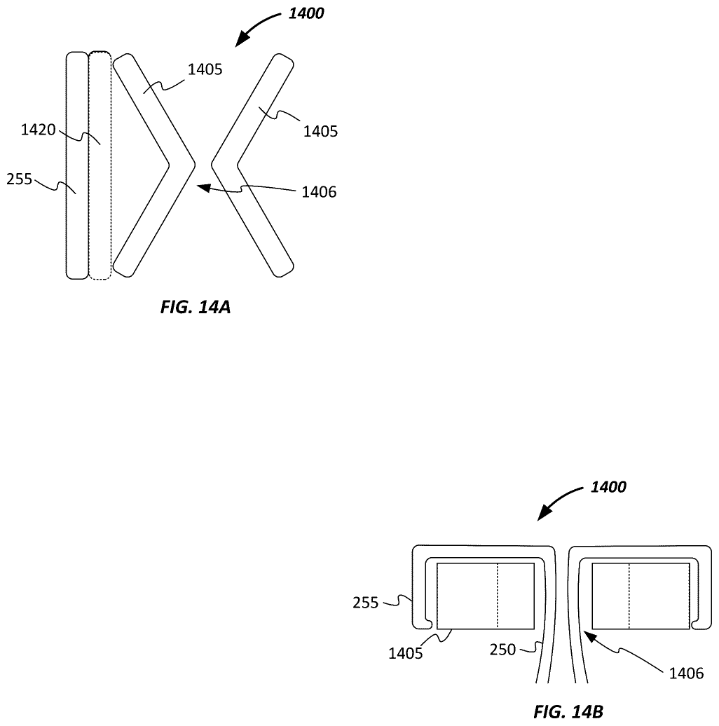

FIG. 14A illustrates a front-view of an X-shaped commissure attachment feature 1400 may include two bent struts 1405 that are unconnected and spaced from one another to define a central slot 1406. In this example, a leaflet attachment region 255 is coupled to commissure attachment feature 1400 so that as the valve closes, the space between the cuff, leaflet and/or stent is reduced. In some examples, the deflection of the commissure attachment feature 1400 may be controlled by the taughtness of the material. As shown in the top view of FIG. 14B, leaflet attachment regions 255 of the leaflets may extend through slot 1406 and wrap around corresponding bent struts 1405 to add compliance to the commissure attachment feature 1400 via a fabric drum effect.

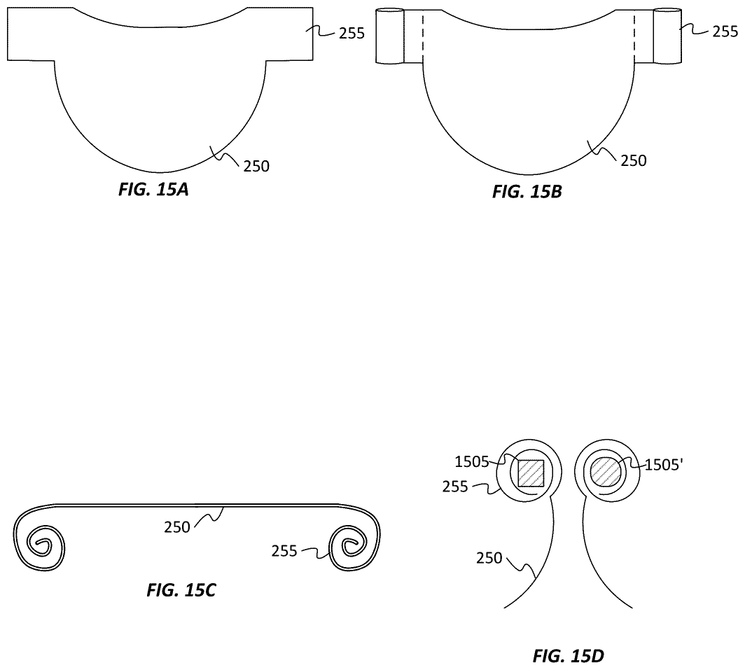

In another embodiment, shown in FIGS. 15A-D, a leaflet 250 may include a pair of extended leaflet attachment regions 255 (FIG. 15A) configured and arranged to be rolled to form a scroll for attachment to a stent (FIGS. 15B-C). As shown in FIG. 15D, the leaflet 250, and specifically leaflet attachment region 255 may be wrapped around the struts of a stent. By way of illustration, in the top view shown in FIG. 15D, two possibilities are presenting including a generally rectangular strut 1505 is shown on the left, and a generally circular or rounded strut 1505′ is shown on the right. It will be understood that either strut (or a combination) may be used in a stent to reduce abrasion on the leaflet attachment regions 255. In this example, as the leaflets close, the scrolls may cinch together and get tighter around the struts.

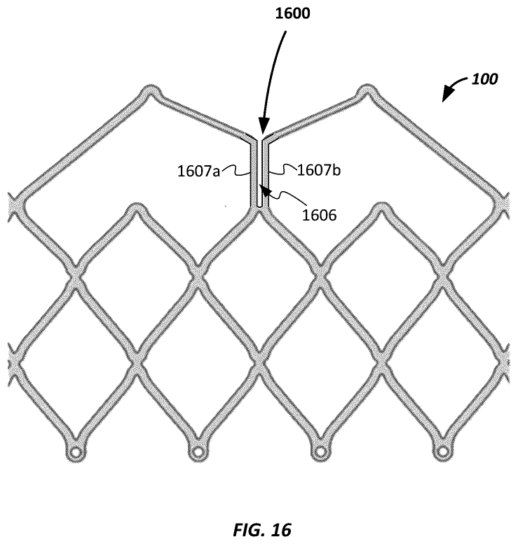

FIG. 16 illustrates an embodiment of a prosthetic heart valve 100 that includes a commissure attachment feature 1160 formed of a pair of spaced parallel beams 1607a, 1607 defining a slot 1606 therebetween. Two leaflets (not shown) may pass through the slot 1606 and be coupled to respective beams 1607a, 1607b using any of the techniques described above, with or without buffering fabric materials. Optionally, the two leaflets may be coupled together as described in previous embodiments. In this example, the two parallel beams 1607a, 1607b are connected at the bottom to one another, but not at the top (e.g., the beams 1607a, 1607b form portions of two side-by-cells that are not connected at the top). Without being bound to any particular theory, it is believed that cantilevering the commissure attachment feature 1160 at the top in this manner will aid in increasing compliance.

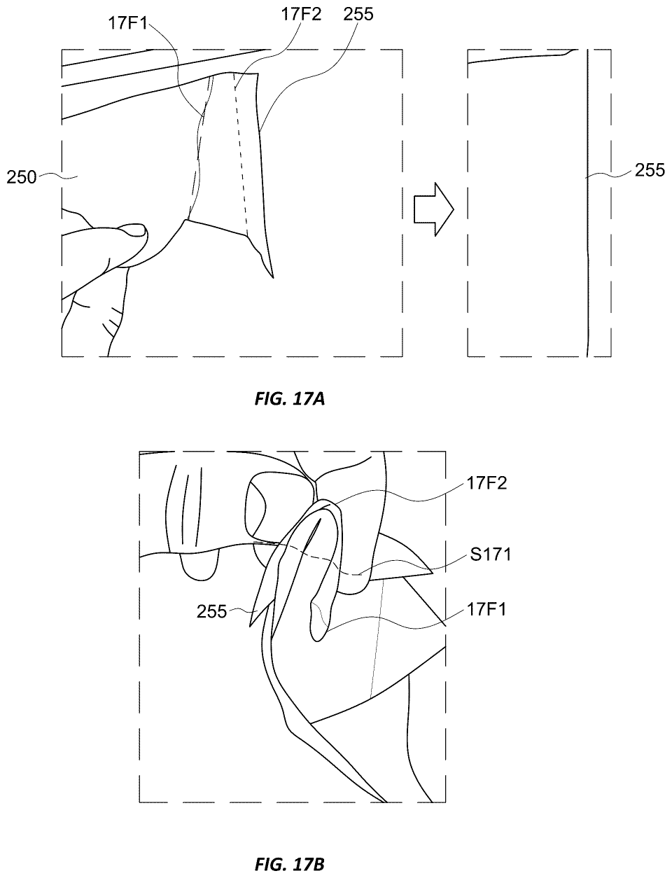

FIGS. 17A-B illustrate a method of folding a leaflet attachment region 255 of a leaflet 250 to achieve greater compliance. In this example, a leaflet attachment region 255 may be initially folded along first foldline 17F1, and then folded along second foldline 17F2 in the opposite direction of the first foldline 17F1, similar to a book fold. A leaflet is shown in the top view of FIG. 17B. In this example, the main leaflet body is buffered on each side by tissue, which protects against abrasion. Optionally a preassembly stitch S171 may lock in the folds and assist in stress distribution. In this manner, leaflet attachment region 255 may be sandwiched between multiple layers of tissue to distribute stress over a larger area.

In use, a prosthetic heart valve may be crimped, loaded and delivered into position, the prosthetic valve having elements that serve to distribute stress and increase compliance. These features may be used to isolate the leaflets, or tabs of the leaflets, and reduce the stress on the valve components during use. It is to be understood that the embodiments described herein are merely illustrative of the principles and applications of the present disclosure. For example, a system combine suturing techniques, commissure attachment features, leaflet attachment regions and/or buffering materials from different embodiments. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve system, comprising:
a stent having a plurality of commissure attachment features;
a cuff coupled to the stent; and
a plurality of leaflets, the cuff and the plurality of leaflets forming a valve assembly, each of the plurality leaflets having two leaflet attachment regions, the leaflet attachment regions being folded adjacent the plurality of commissure attachment features.

2. The prosthetic heart valve system of claim 1, wherein each of the plurality of commissure attachment features includes an elongated commissure attachment feature having a plurality of eyelets, and further comprising a designated suture for coupling each of the leaflet attachment regions to a respective commissure attachment feature through ones of the plurality of eyelets.

3. The prosthetic heart valve system of claim 1, wherein each of the plurality of commissure attachment features includes an elongated commissure attachment feature having a plurality of longitudinally extend slots.

4. The prosthetic heart valve system of claim 3, wherein the plurality of longitudinally extend slots includes two slots.

5. The prosthetic heart valve system of claim 4, further comprising a rectangular reinforcing swatch disposed over each of the plurality of commissure attachment features.

6. The prosthetic heart valve system of claim 5, further comprising a suture for coupling each of the leaflet attachment regions to the rectangular reinforcing swatch.

7. The prosthetic heart valve system of claim 6, wherein the suture comprises a first running stitch pattern from a proximal end to a distal end and a second reverse-running stitch pattern from the distal end to the proximal end.

8. The prosthetic heart valve system of claim 3, wherein the plurality of longitudinally extend slots includes three slots.

9. The prosthetic heart valve system of claim 8, wherein each of the plurality of commissure attachment features is coupled by two upper struts and two lower struts.

10. The prosthetic heart valve system of claim 8, wherein each of the leaflet attachment regions passes through a central one of the three slots.

11. The prosthetic heart valve system of claim 1, wherein each of the plurality of commissure attachment features includes a Π-shaped commissure attachment feature having two side beams and an upper beam.

12. The prosthetic heart valve system of claim 11, further comprising a horseshoe-shaped scroll of fabric covering the side beams and the upper beam.

13. The prosthetic heart valve system of claim 1, wherein each of the plurality of commissure attachment features comprises a pair of spaced parallel beams coupled together at lower ends, but not at upper ends.

14. The prosthetic heart valve system of claim 1, wherein each of the leaflet attachment regions are rolled to form a scroll over a portion of a respective commissure attachment feature.

15. A prosthetic heart valve system, comprising:
a stent having a plurality of commissure attachment features;
a cuff coupled to the stent and having a plurality of longitudinal slits; and
a plurality of leaflets, the cuff and the plurality of leaflets forming a valve assembly, each of the plurality leaflets having two leaflet attachment regions, the leaflet attachment regions passing through respective longitudinal slits in the cuff.

16. The prosthetic heart valve system of claim 15, further comprising an outer second fabric disposed on an abluminal surface of the leaflet attachment regions.

17. The prosthetic heart valve system of claim 15, wherein the cuff is an inner fabric disposed on a luminal surface of the stent.

18. The prosthetic heart valve system of claim 15, wherein the cuff is an inner fabric disposed on an abluminal surface of the stent.
